(19) United States
(12) Patent Application Publication
(10) Pub. No.: US 2008/0206103 A1
(43) Pub. Date: Aug. 28, 2008

(54) Method For The Preparation Of Very Stable, Self-Assembled Monolayers On The Surface Of Gold Coated Microcantilevers For Application To Chemical Sensing

(75) Inventors: Lal A. Pinnaduwage, Knoxville, TN (US); Thomas G. Thundat, Knoxville, TN (US); Gilbert M. Brown, Knoxville, TN (US); Peter V. Bonnesen, Knoxville, TN (US); Vassil Boiadjiev, Philadelphia, PA (US); Gudron Goretzki, Nottingham (GB)

Correspondence Address: BOYLE FREDRICKSON S.C., 840 North Plankinton Avenue, MILWAUKEE, WI, 53203, US

(73) Assignees: UT-BATTELLE, LLC, Oak Ridge, TN (US); UNIVERSITY OF TENNESSEE RESEARCH FOUNDATION, Knoxville, TN (US)

(21) Appl. No.: 11/662,696

(22) PCT Filed: Sep. 14, 2005
(86) PCT No.: PCT/US05/32637

§ 371 Date: Mar. 10, 2008

Related U.S. Application Data
(63) Continuation-in-part of application No. 11/152,627, filed on Jun. 14, 2005, now abandoned.
(60) Provisional application No. 60/609,610, filed on Sep. 14, 2004.

(51) Int. Cl. B01J 19/00; C07D 323/00; C07C 43/215

(52) U.S. Cl. 422/82.01; 549/348; 568/633; 422/68.1; 422/82.05

(57) ABSTRACT

Methods for the preparation of a stable, self-assembled monolayer on the silicon surface or metallic coating of a microcantilever are disclosed. The methods produce a microcantilever suitable as a chemical sensor. In a microcantilever produced using one version of the method, a metallic coating is disposed on a side of the microcantilever, a bridging atom is bonded to the metallic coating, a first spacer group is bonded to the bridging atom, a second spacer group is bonded to the bridging atom, and a chemical recognition agent is bonded to the first spacer group. In another version of the method, a silicon surface of a microcantilever is hydrogen terminated, and a calixarene chemical recognition agent is carbon linked to the silicon surface using photochemical hydrosilylation. Among other things, the calixarene may be bonded to a crown ether for ion detection or bonded to a area for the recognition of explosives by hydrogen bonding to nitro groups.

### CROSS-REFERENCES TO RELATED APPLICATIONS

This application claims priority to U.S. Provisional Patent Application No. 60/609,610 filed Sep. 14, 2004 and claims the benefit of U.S. patent application Ser. No. 11/152,627 filed Jun. 14, 2005.

### STATEMENT REGARDING FEDERALLY SPONSORED RESEARCH

This invention was made with United States Government support under Contract No. DE-AC05-00OR22725 between the United States Department of Energy and awarded to U.T. Battelle, LLC. The United States Government has certain rights in this invention.

### BACKGROUND OF THE INVENTION

1. Field of the Invention

This invention relates to methods for the preparation of stable, self-assembled monolayers on the silicon surface or gold surface of gold coated microcantilevers. The microcantilevers with the stable, self-assembled monolayer can be used in chemical sensing applications.

2. Description of the Related Art

General design parameters are known for constructing chemical sensors for the detection of analytes at very low concentrations. These chemical sensors selectively concentrate species of interest on a surface using a chemical agent for molecular recognition. A molecular recognition agent is incorporated in a matrix that enhances selectivity (e.g., polymer film, polymer beads, chemically modified surface). A transduction mechanism is used to recognize that the analyte of interest has been captured (e.g., electrochemistry, fluorescence, microcantilever, Raman spectroscopy). Preferably, there is an array of sensor elements for “cross-selectivity”, and electronic readout and communication of the results.

The development of a new platform of miniature sensors for chemical and physical properties based on microelectromechanical systems (MEMS) technology is beginning to be realized. In 1994, researchers observed that the microcantilevers used in atomic force microscopy (AFM) were quite sensitive to external physical and chemical influences. For example, Thundat et al. pointed out the possible use of bending and frequency shift in microcantilevers for chemical sensing (see, Chen et al., “Adsorption-Induced Surface Stress and Its Effects on Resonance Frequency of Microcantilevers,” Journal of Applied Physics, vol. 77, no. 8, pp. 3618-3622, 1995; Thundat et al., “Thermal and Ambient-Induced Deflections of Scanning Force Microscope Cantilevers,” Applied Physics Letters, vol. 64, no. 21, pp. 2894-2896, 1994; Thundat et al., “Detection of Mercury-Vapor Using Resonating Microcantilevers,” Applied Physics Letters, vol. 66, no. 13, pp. 1695-1697, 1995; and Thundat et al., “Vapor Detection Using Resonating Microcantilevers,” Analytical Chemistry, vol. 67, no. 3, pp. 519-521, 1995). Gimzewski et al. pointed out possible applications in thermal calorimetry (see, Barnes et al., “Photothermal Spectroscopy with Femtojoule Sensitivity Using a Micromechanical Device,” Nature, vol. 372 pp. 79-81, 1994; and Barnes et al., “A Femtojoule Calorimeter Using Micromechanical Sensors,” Review of Scientific Instruments, vol. 65, no. 12, pp. 3793-3798, 1994). Since then researchers all over the world have been reporting the use of silicon or silicon nitride microcantilevers for a variety of sensing applications. Microcantilevers are fabricated with the length of the cantilevers often in the range of 100-200 microns while the thickness ranges from 0.3-1 micron. Recent advances in micromachining allow the fabrication of cantilever beams that can detect extremely small forces and mechanical stresses, promising to bring about a revolution in the field of chemical, physical, and biological sensor development. The key to the high sensitivity of the microcantilevers is the enormous surface-to-volume ratio, which leads to amplified surface stress.

Microcantilevers have two main signal transduction methods: bending and mass-loading. In the mass-loading mode, microcantilevers behave just like other gravimetric sensors such as quartz crystal microbalances and surface acoustic wave (SAW) transducers, that is, their resonance frequencies decrease due to the adsorbed mass. This adsorption can be enhanced for a particular analyte by coating both broad surfaces of a microcantilever with a specific chemical. The chemical coatings provide enhanced detection as well as a degree of selectivity. The other signal transduction method, i.e., the bending response, is unique to the microcantilever—if a differential surface stress is achieved, for example, by using a coating on one of its broad surfaces, the microcantilever will bend. Since a differential surface stress is required for bending, only one broad surface should be coated for bending mode operation. Because the bending mode has been shown to be more sensitive compared to the mass-loading mode, normally the coating is applied only to one broad surface of the microcantilever. Therefore, the mass-loading information is used only as a bonus. In the bending mode, microcantilever detection sensitivity is at least an order of magnitude higher than other miniature sensors such as quartz crystal microbalances and surface acoustic wave transducers that are also being investigated as chemical sensors.

To understand how sensitive sensors are created with microcantilevers it should be appreciated that the bending of the microcantilever is not due to the weight of the deposited material. A 40-ng microcantilever bends about 1 nanometer due to its own weight, which is just above the noise level for a cantilever-bending signal. Therefore, the microcantilever bending due to the weight of the deposited material of pico-gram levels is insignificant. On the other hand, for micron-size objects like microcantilevers, the surface-to-volume ratio is large and the surface effects are enormously magnified. Thus adsorption-induced surface forces can be extremely large. The adsorption-induced force can be viewed as due to change in surface free energy due to adsorption. Free energy density (mJ/m²) is the same as surface stress (N/m), and surface stress has the units of the spring constant of a cantilever. Therefore, if the surface free-energy density change is comparable to the spring constant of a cantilever, the cantilever will bend. When probe molecules bind to their targets, steric hindrance and electrostatic repulsions cause the bound complexes to move apart. Because they are tethered at one end and because the surface area is finite, they exert a force on the surface.

Another advantage of the microcantilever sensor platform is that it works with ease in air and in liquid. Both resonance frequency and bending modes can be used in liquid. Due to the small mass of microcantilevers they exhibit thermal motion (Brownian motion) in air and liquid. Therefore, no external excitation technique is needed for exciting cantilevers into resonance. The bending of the cantilever can be detected by a variety of methods that have been developed for atomic force microscopy, i.e. optical, piezoresistive, piezoelectric, electron tunneling, and capacitive methods (see, e.g., Sarid, “Scanning force microscopy with applications to electric, magnetic, and atomic forces”, New York: Oxford University Press, 1991). The resonance frequency of the cantilever can be detected by feeding the bending signal to a spectrum analyzer.

Despite its high sensitivity, the cantilever platform offers no intrinsic chemical selectivity. One surface of the silicon microcantilever can be functionalized so that a given molecular species will be preferentially bound to that surface upon its exposure to an analyte stream. Therefore, detection sensitivity is vastly enhanced by applying an appropriate coating on one cantilever surface. Such a coating can, in principle, provide selectivity as well.

Selectivity and reversibility are often competing characteristics of chemical sensors. The type of interaction occurring between analyte molecules and the cantilever coating determines the adsorption and desorption characteristics. Low-energy, reversible interactions such as physisorption generally lack an acceptable degree of selectivity, that is, the energies involved range from van der Waals interactions (energy ˜0-10 kJ mol⁻¹) to acid base interactions (energy<40 kJ mol⁻¹). Furthermore, the weak interaction may lead to insufficient sorption, making sensor response weak. At the other end of the spectrum, highly selective interactions form strong bonds that are normally covalent in nature (chemisorption) and are not reversible (binding energies are 300 kJ mol⁻¹) under normal conditions.

There are two “intermediate-range” interactions that can be considered to provide limited selectivity while being reversible. One is hydrogen bonding and the other is coordination chemistry. A hydrogen bond is formed by one hydrogen atom and two electronegative atoms, one of which is covalently bound to the hydrogen atom. For example, the oxygen atoms in the characteristic nitro groups of explosives can participate in hydrogen bonding.

A coordination compound consists of a central metal atom surrounded by neutral or charged, often organic, ligands. In the ligand, one or more donor atoms interact with the metal ion. The selectivity now can be influenced by the choice of the metal ions as well as by the choice of the ligand, both from an electronic or steric point of view (see, Nieuwenhuizen et al., “Processes Involved at the Chemical Interface of a SAW Chemosensor,” Sensors and Actuators, vol. 11, no. 1, pp. 45-62, 1987). Some of the well established principles of molecular recognition can be used to advantage in the design of ligands. In most applications, it is desirable to have the ability to regenerate the sensor, and thus the use of “intermediate range” interactions will be necessary, which in turn broadens the target range. Therefore, normally a single microcantilever coating does not provide sufficient selectivity if reversible sensor operation is required. In general, it will be necessary to use an array of microcantilevers with multiple coatings in order to obtain sufficient selectivity especially if the sensor is required to monitor multiple analytes. Pattern recognition schemes (using neural analysis) needs to be employed to extract the composition of the target stream.

Many chemically selective coatings for chemical speciation have also been developed. Receptor-ligand, antibody-antigen, or enzyme-substrate reactions have been studied for biological detection. Advances have also been made in many other crucial areas such as immobilization of selective agents on cantilever surfaces, and application of selective layers on cantilever arrays. Aided by such tools, physical, chemical, and biological detection have been demonstrated using microcantilever sensors. These developments together with the recent advances in neural analysis and telemetry pave the way to the development of smart, miniature sensors.

Cantilevers undergo bending due to molecular adsorption when adsorption is confined to a single side of the cantilever. Microcantilever deflection varies sensitively as a function of adsorbate coverage. Microcantilevers, such as those having a thin coating of gold on one side that are used in the following experiments, have an intrinsic deflection due to unbalanced stresses on the opposing surfaces. Although bending can be expected for films of many atomic layers due to differences in physical parameters such as elastic and lattice constants, bending due to submonolayer coverage as small as 10⁻³ monolayers is not intuitive. One monolayer of a gold surface has 1.5×10¹⁵ atoms/cm² while on a Si(111) surface, the density is 7.4×10¹⁴ atoms/cm². Therefore, 10⁻³ monolayers corresponds to approximately 10¹² atoms on the surface of the cantilever.

Using Stoney's formula and equations of bending of a cantilever, a relation can be derived between the cantilever bending and changes in surface stress. Since one does not know the absolute value of the initial surface stress, one can only measure the variation in surface stress. The surface stress variation between top and bottom surface of a cantilever can be written as:

Δσ₁−Δσ₂=(zEt²)/(4L²(1−v))

where, z is the cantilever deflection, E is the Young's modulus, L is the cantilever length, t is the thickness and v is the Poisson ratio. Since all the quantities on the right hand side can be measured (or known apriori), the changes in surface stress due to adsorption can be calculated.

Surface stress, σ, and surface free energy, γ, can be related using the Shuttleworth equation: σ=γ+(δγ/δε), where σ is the surface stress (see, Shuttleworth, “The Surface Tension of Solids,” Proc. Phys. Soc. (London), vol. 63A pp. 444-457, 1950). The surface strain δε is defined as the ratio of change in surface area, δε=dA/A. Since the bending of the cantilever is very small compared to the length of the cantilever, the strain contribution is only in the ppm (10⁻⁶) range while the surface free energy changes are in the 10⁻³ range. Therefore, one can easily neglect the contribution from surface strain effects and equate the free energy change to surface stress variation (see, Butt, “A sensitive method to measure changes in the surface stress of solids,” Journal of Colloid and Interface Science, vol. 180, no. 1, pp. 251-260, 1996).

Investigations of chemical and physical sensing with microcantilever-based devices have been conducted to date. For example, Thundat and co-workers have developed microcantilever-based sensors for a number of species based on alkanethiol reagents sorbed to a microcantilever coated with gold on one surface. A calix[4]arene crown-6-ether as an alkanethiol derivative is selective for Cs⁺ ions (see, Ji et al., “A novel self-assembled monolayer (SAM) coated microcantilever for low level caesium detection”, Chemical Communications, no. 6, pp. 457-458, 2000). Quaternary ammonium and pyridinethiol SAMS were shown to be selective for Cr(VI) (see, Ji et al., “Ultrasensitive detection of CrO₄²⁻ using a microcantilever sensor,” Analytical Chemistry, vol. 73, no. 7, pp. 1572-1576, 2001; and Pinnaduwage et al., “Detection of Hexavalent Chromium in Ground Water Using a Single Microcantilever Sensor,” Sensor Letters, 2004). Gold is itself selective for Hg(0) in the vapor phase and in solution and for Hg(II) in solution (see, Xu et al., “Detection of Hg²⁺ using microcantilever sensors,” Analytical Chemistry, vol. 74, no. 15, pp. 3611-3615, 2002). A SiO₂ cantilever is selective for HF and F⁻ (see, Tang et al., “Detection of Femtomole HF Using a SiO₂ Microcantilever,” Journal of the American Chemical Society, 2004). A coating of L-cysteine is selective for Cu(II) binding (see, Xu et al., “Ultrasensitive Detection of Cu²⁺ Using a Microcantilever Sensor Modified with L-Cysteine Self-Assembled Monolayer”, 2004). This latter Cu(II) coordinated surface was shown to be selective for the nerve agent stimulant, dimethylmethylphosphonate (see, Yang et al., “Nerve agents detection using a Cu²⁺/L-cysteine bilayer-coated microcantilever,” Journal of the American Chemical Society, vol. 125, no. 5, pp. 1124-1125, 2003).

Some of these microcantilever-based sensors do have limitations. For example, the self-assembled monolayer of some of these sensors may not be stable over an acceptable period of time. Thus, there is a need for improved methods for the preparation of stable, self-assembled monolayers on the surface of gold coated microcantilevers so that the microcantilevers can be used in chemical sensing applications.

### SUMMARY OF THE INVENTION

The foregoing needs are met by the present invention which provides microcantilever sensors with chemical selectivity. Microcantilever-based sensors have been shown to be extremely sensitive, however silicon or silicon nitride microcantilevers coated on one surface with gold do not have any particular chemical selectivity. Chemical selectivity has been achieved by coating the gold surface of the microcantilevers with a selective film such as a self-assembled monolayer (SAM) of a thiol having a head group suitable for molecular recognition. The approach of the present invention to the design of selective sensors is to immobilize agents for selective molecular recognition in a matrix that mimics the organic medium in a solvent extraction system. In this manner, the matrix can enhance both the separation and the achievement of chemical selectivity. The transduction part of the microcantilever sensor is based on binding the molecular recognition agent to one surface of the cantilever so that the adsorption-induced stress change can be detected via bending of the microcantilever.

Calix[4]arenes have been widely used as a three-dimensional platform for selective molecular recognition. This invention is a new way to attach these calix[4]arenes in the 1,3-alternate confirmation to the surface of cantilevers. It has been shown that calix[4]arene-crown-6 ethers in the 1,3-alternate conformation bind cesium with remarkable strength and selectivity, and this was the basis of a microcantilever sensor for Cs⁺ in solution (see, Ji et al., “A novel self-assembled monolayer (SAM) coated microcantilever for low level caesium detection”, Chemical Communications, no. 6, pp. 457-458, 2000). New chemistry has been developed for the attachment of SAMs of calix[4]arenes in the 1,3-alternate conformation as dialkanesulfides. This attachment has been shown to form SAMs that are stable for a period of over a month in solution. The 2,4-arene rings allow for attachment of molecular recognition groups in a well defined geometry. In addition to the attachment of crown ethers for metal ion separation, ureas and thioureas have been attached for recognition of explosives by hydrogen bonding to nitro groups, and cationic ion exchangers for recognition of perchlorate by selective ion exchange. This invention is a general synthetic scheme for the preparation of a variety of head groups to calixarenes and the attachment chemistry to cantilevers as both dialkanesulfides and as silane reagents.

These and other features, aspects, and advantages of the present invention will become better understood upon consideration of the following detailed description, drawings, and appended claims.

### BRIEF DESCRIPTION OF THE DRAWINGS

FIG. 1 shows the formation of a self-assembled monolayer on a gold coated cantilever using a thiol or a disulfide.

FIG. 2 shows the dialkylsulfide chemistry according to the invention being used with a quaternary ammonium head group.

FIG. 3 shows four possible isomeric conformations of calix[4]arenes suitable for use with the present invention.

FIG. 4 shows 1,3-alternate 25,27-bis(11-mercapto-1-undecanoxy)-26,28-calix[4]benzocrown-6 (1) that has been used for the microcantilever sensing of cesium ions.

FIG. 5 shows an example general method for the preparation of 1,3-alt-calix[4]arenes with two dialkylsulfide attachment sites for self-assembled monolayer formation where X is a leaving group and R is a molecular recognition group.

FIG. 6 shows an example sensor according to the invention.

FIG. 7 shows bending signal versus time for a microcantilever and demonstrates that response signals are very reproducible.

FIG. 8 shows the preparation of calix[4]arene-urea derivatives according to the invention for the selective adsorption of explosives containing nitro-groups.

FIG. 9 shows a synthetic strategy for preparing 25,27-bis[11 (mercaptodecyl)undecyloxy]calix[4]arene-crown-5 (6) and 25,27-bis[11 (mercaptodecyl)undecyloxy]calix[4]arene-crown-6 (7).

FIG. 10 shows photochemical hydrosilylation of alkenes and alkynes. FIG. 10 is taken from Buriak, Chem. Rev. 2002,102 (5), 1271-1308.

FIG. 11 shows a scheme for attaching a siloxane reagent to an olefin group on a calixarene.

### DETAILED DESCRIPTION OF THE INVENTION

The modification of gold surfaces by self-assembled monolayers is a versatile method of immobilizing a substrate in an ordered, densely packed arrangement and thereby influencing the physical properties of the surface. The introduction of recognition units allows the development of rapidly responding sensors. Monolayer based sensing devices should incorporate these properties. The existence of a specific and reversible recognition based on a supramolecular framework allows interaction between the analyte and the monolayer. In addition the binding of the analyte has to result in a signal transduction.

This invention provides two different methods of attaching chemical recognition agents to the surface of a cantilever coated with metal, such as gold, on one surface to make the cantilevers chemically selective. Chemical recognition agents including calix[4]arenes in the 1,3 alternate conformation are an example agent. The beauty of these molecules lies in the easy opportunity to modify the two different sites independently, easily and selectively. Organosulfur compounds bind strongly to gold, and SAMs of complex molecules, e.g., tetra sulfane functionalized calixarenes and resorcinarenes have been reported (see, Schoenherr et al., Langmuir 1997, 13, 1567; Schoenherr et al., Langmuir 1999, 15, 5541; van Velzen et al., J. Am. Chem. Soc., 1994, 116, 3597; and Faull et al., Langmuir 2002, 28, 6585). Di-functionalized calix[4]arene compounds give the opportunity for incorporation of additional recognition units in contrast to the previously reported experiments. The introduction of two additional alkyl chains leads to a significant stabilization of the calixarene based monolayers, due to improved van der Waals interaction of the proximate chains and makes the thiol co-absorption redundant. The use of a single reagent also prevents phase separation and provides additional stabilization.

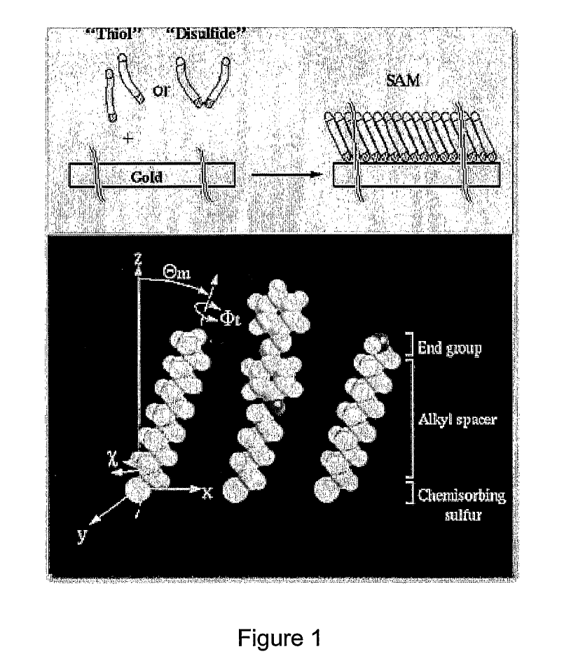

FIG. 1 shows the formation of a self-assembled monolayer on a gold coated cantilever. A cantilever is coated on one surface with gold. Alkane thiols and alkyl disulfides can form self-assembled monolayers on the gold surface, and the head (end) group allows for molecular recognition. The stability of the monolayer is due to gold-sulfur covalent bonding as well as van der Waals interactions between chains. Long alkyl chains work together to align neighboring molecules into a collective orientation.

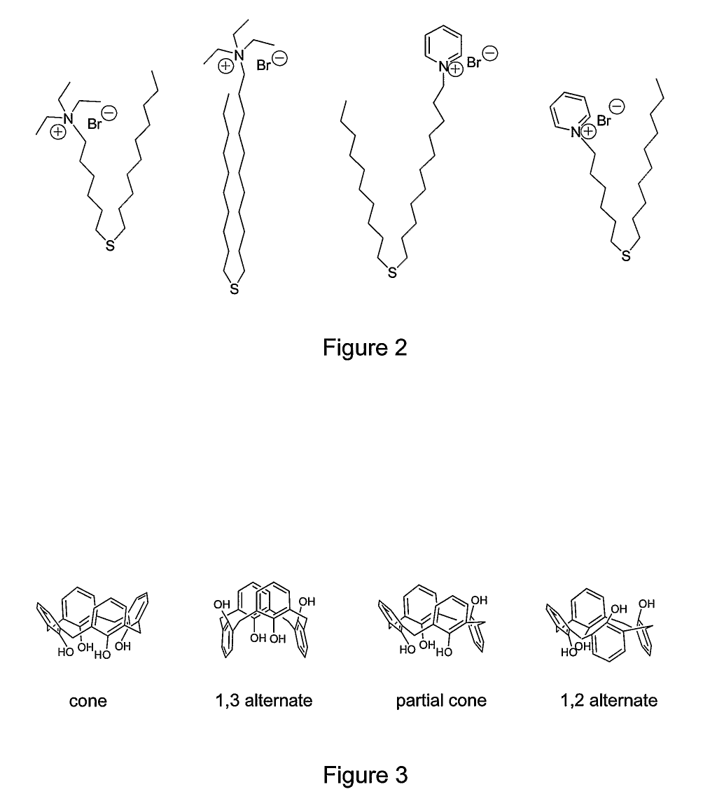

To allow interaction with the gold surface, in one embodiment, one site of the molecule is functionalized with dialkylsulfide chains, using the Anti-Markivnikov addition of alkylthiols to double bonds in the presence of 9-borabicyclononane (9-BBN). The compounds are able to organize in stable self assembled monolayers on gold surfaces, which is crucial for reproducibility, sensibility and selectivity of the recognition process. The “upper” site can be functionalized according to the chemical recognition problem. Thus, dialkylsulfides form stable self-assembled monolayers. The chemistry for formation of dialkanesulfides is compatible with a wider variety of molecular recognition elements. For example, FIG. 2 shows that the dialkylsulfide chemistry has solved the problem of instability of quaternary ammonium head groups.

With respect to the chemical recognition sites, calix[4]arenes are an advantageous three-dimensional framework for chemical recognition sites. FIG. 3 shows four possible isomeric conformations of calix[4]arenes. The 1,3-alt conformation is preferred in the present invention as it has most flexibility for modification, that is, the OH groups on the two sides of the calix[4]arene molecule can be substituted independently and selectively and are easily accessible compared to the 1,2-alt confirmation. To allow interaction with the gold surface, one or two sites are functionalized on one side of the molecule with dialkylsulfide chains. The “upper” sites can then be functionalized for chemical recognition.

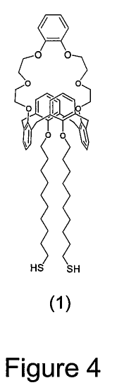

In particular, calix-crown-6 ethers in the 1,3 alternate conformation are valuable compounds for cesium extraction from highly radioactive waste (see, e.g., U.S. Pat. No. 6,174,503). Work has been performed regarding the synthesis and the application to microcantilever sensing of Cs⁺ using 1,3-alternate 25,27-bis(11-mercapto-1-undecanoxy)-26,28-calix[4]benzocrown-6 (1) (see FIG. 4) in a mixed monolayer with decanethiol (see, Ji et al., Chem. Commun. 2000, 457). Due to difficulties in the reproducibility of this obtained data, improvement of the stability of the monolayers was achieved by going to the dialkylsulfide attachment chemistry of the present invention. The new crown compounds differ from the ligand previously investigated in that dialkanethiol groups are employed instead of alkanethiols to eliminate the need to co-adsorb decanethiol to fill in the gaps between the calixarene ionophores.

The attachment chemistry of the present invention is also compatible with other chemical recognition agents including quaternary ammonium and pyridine groups (chromate selective), crown ethers and azacrown compounds (metal ion selective), borate esters (sugars), ureas and thioureas (nitrate and organonitrate compounds), biomolecule-selective antibody-antigens, DNA, proteins, as well as organic acids, esters, amides, amines, aldehydes, phosphonic acids and esters, buckyballs, hydroxyls, and related compounds.

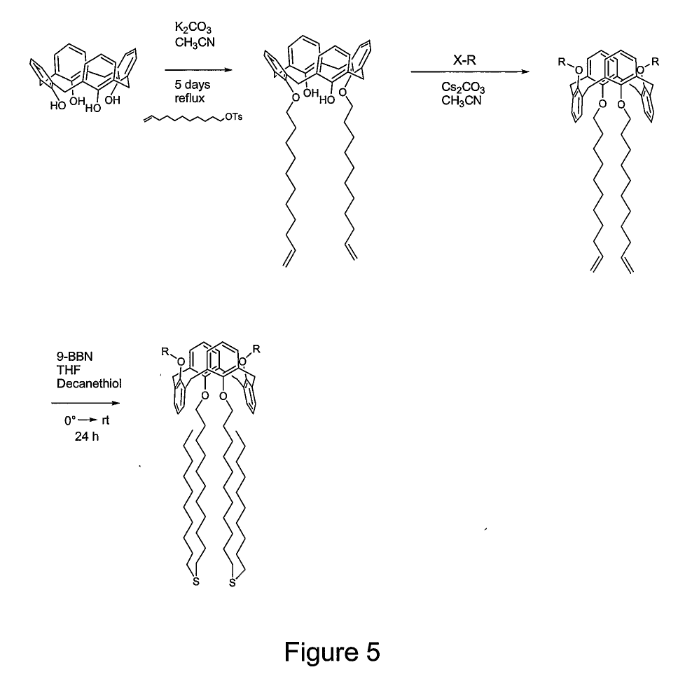

FIG. 5 shows an example general method for the preparation of 1,3-alt-calix[4]arenes with two dialkylsulfide attachment sites for self-assembled monolayer formation where X is a leaving group and R is a molecular recognition group, and 9-BBN is 9-borabicyclo[3.3.1]nonane.

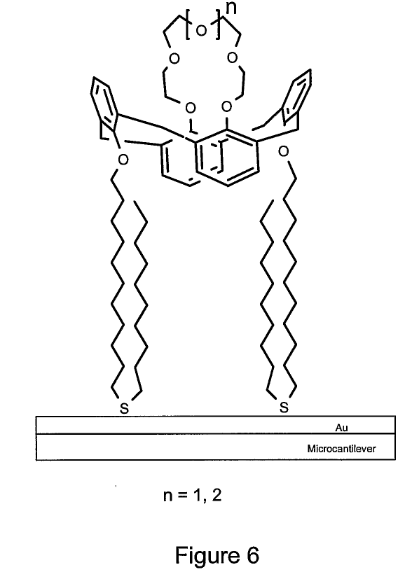

One non-limiting example of an application of a microcantilever sensor including a self-assembled monolayer according to the invention is the detection of metal ions in aqueous solution using principles of molecular recognition. For example, the invention provides a sensor for metal ions needed for monitoring cleanup following a “dirty bomb” (a conventional explosive to spread radioactive ions over a wide area), a sensor to monitor drinking water, sensors for chemical weapons agents and TICs in water. The selective complexing agent for the metal ion of interest can be attached to the 2,4-sites on a calix[4]arene. Another example sensor according to the invention is shown in FIG. 6. Radioactive Cs⁺ can be a problem in tank waste. Previous results at a sensor have been irreproducible due to the requirement for formation of a mixed monolayer. The use of a dialkylsulfide reagent in accordance with the invention does not require a co-adsorbant for stability. In one form, one week is required for formation of monolayer which is stable for three months. In FIG. 6, the n=2 form is Cs⁺ selective, and the n=1 form is more selective for K⁺.

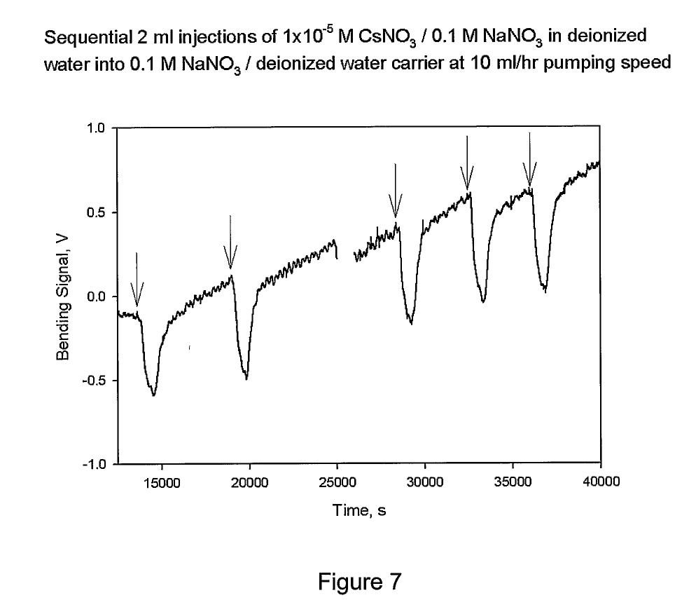

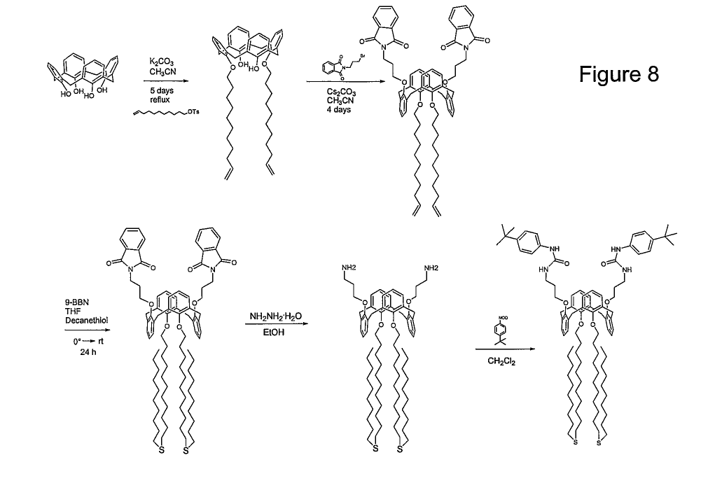

FIG. 7 shows that microcantilever response signals are very reproducible. The same cantilever is reused by recycling with 0.1 M NaCl solution. NaCl recycles cantilever faster than NaNO₃, and the signals are reversible in 0.1 M NaNO₃ background electrolyte. Thus, the binding interaction is reversible using electrolytecycling.

FIG. 8 shows the preparation of calix[4]arene-urea derivatives for selective adsorption of explosives containing nitro-groups. It has been shown that low vapor pressure nitro compounds can be detected by extraction from soil into an organic solvent followed by high performance liquid chromatography: acetone or acetonitrile for extraction from soil. In the present invention, a liquid chromatograph is replaced with an array of microcantilevers, each optimized to an explosive compound. The explosive is extracted from swiping pad with organic solvent and then detected with an array of microcantilever sensor elements.

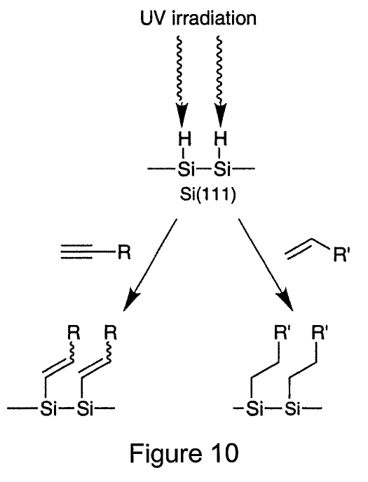

In an alternative attachment method according to the invention, photochemical hydrosilylation can be used for the direct covalent attachment of the calixarenes via robust Si—C bonds to the silicon surface of a microcantilever. This can be achieved in a two-step process involving hydrogen termination of the silicon cantilever surface and subsequent photochemical (UV) hydrosilylation with unsaturated terminal vinyl groups of the calixarene, which results in stable Si—C surface linkages (see a general reaction scheme in FIG. 10).

Accordingly, the invention provides an improved chemical sensor having one or more microcantilevers with a stable self-assembled monolayer. The microcantilever(s) have a metallic coating disposed on a side of the microcantilever. A bridging atom is bonded to the metallic coating. A first spacer group is bonded to the bridging atom and also to a chemical recognition agent for detecting a species of interest (e.g., an atom, a molecule or an ion). A second spacer group is bonded to the bridging atom and fills gaps present between any adjacent first spacer groups.

The metallic coating on the microcantilever(s) may comprise a metal selected from the group consisting of gold, platinum, copper, palladium, aluminum and titanium, and preferably, the metallic coating comprises gold. The bridging atom should preferably chemically bond to the metallic coating for stability, and sulfur is a preferred bridging atom as it covalently bonds with gold, the preferred metallic coating.

The method of preparation dictates that the first spacer group is preferably selected from the group consisting of unsubstituted or substituted alkylene groups, unsubstituted or substituted alkenylene groups, or unsubstituted or substituted alkynylene groups. Most preferably, the first spacer group is selected from C₅-C₂₅ alkylene groups. One non-limiting example alkylene group is undecylene.

The second spacer group is preferably selected from the group consisting of unsubstituted or substituted alkyl groups, unsubstituted or substituted alkenyl groups, or unsubstituted or substituted alkynyl groups. Most preferably, the second spacer group is selected from C₅-C₂₅ alkyl groups. One non-limiting example alkyl group is decyl.

The chemical recognition agent for detecting a species of interest (e.g., an atom, a molecule or an ion) may be a chemical recognition agent selected from quaternary ammoniums, pyridines, crown ethers, azacrown compounds, borate esters, ureas, thioureas, antibody-antigens, organic acids, organic esters, organic amides, organic amines, organic aldehydes, phosphonic acids, phosphonic esters, buckyballs, and hydroxyls. The chemical recognition agent is selected based on the atom, molecule or ion that one wishes to detect. For example, the chemical recognition agent may be a calixarene bonded to a crown ether for ion detection, a calixarene bonded to a urea or thiourea for nitro group-containing explosive detection, or a calixarene bonded to a cationic ion exchanger for anion detection.

The chemical sensor includes means for detecting a binding interaction between the chemical recognition agent and the atom, the molecule or the ion. For example, the means for detecting the binding interaction may comprise at least one method selected from the group consisting of optical, piezoresistive, piezoelectric, and capacitive. For example, the bending of a microcantilever can measured by monitoring the position of a laser beam reflected off the top of the microcantilever onto a four-quadrant photodiode. Preferably, the binding interaction is reversible using electrocycling or electrolytecycling so that the chemical sensor may be reused. In one form, the binding interaction causes a change in surface stress in the microcantilever.

The chemical sensor preferably includes an array of microcantilevers, and at least some of the microcantilevers include a metallic coating disposed on a side of the microcantilever, a bridging atom bonded to the metallic coating, a first spacer group bonded to the bridging atom and a chemical recognition agent for detecting an atom, a molecule or an ion, and a second spacer group bonded to the bridging atom. Some of the microcantilevers in the array may have different chemical recognition agents, and one or more reference microcantilevers may be in the array to provide means to eliminate background signals from cantilever bending. For example, the reference microcantilevers may lack the chemical recognition agent.

The invention also provides another improved chemical sensor having one or more microcantilevers with a stable self-assembled monolayer. The microcantilever has a silicon surface and a surface having a metallic coating. A spacer group is bonded to the silicon surface, and a chemical recognition agent comprising a calixarene for detecting an atom, a molecule or an ion is bonded to the spacer group. The metallic coating may be a metal selected from the group consisting of gold, platinum, copper, palladium, aluminum and titanium, and preferably is gold. Preferably, the chemical recognition agent comprises a calixarene bonded to a group selected from crown ethers, ureas, thioureas, and cationic ion exchangers.

The method of preparation dictates that the spacer group is preferably selected from the group consisting of unsubstituted or substituted alkylene groups, unsubstituted or substituted alkenylene groups, or unsubstituted or substituted alkynylene groups. Most preferably, the first spacer group is selected from C₅-C₂₅ alkylene groups. One non-limiting example alkylene group is undecylene. The spacer group may include a siloxane group bonded to the silicon surface.

The chemical sensor includes means for detecting a binding interaction between the chemical recognition agent and the atom, the molecule or the ion. For example, the means for detecting the binding interaction may comprise at least one method selected from the group consisting of optical, piezoresistive, piezoelectric, and capacitive. Preferably, the binding interaction is reversible using electrocycling or electrolytecycling so that the chemical sensor may be reused. In one form, the binding interaction causes a change in surface stress in the microcantilever.

The chemical sensor preferably includes an array of microcantilevers, and some of the microcantilevers in the array may have different chemical recognition agents, and one or more reference microcantilevers may be in the array to provide means to eliminate background signals from cantilever bending. For example, the reference microcantilevers may lack the chemical recognition agent.

One method of the invention uses a calixarene that may be bonded to the metallic coating on the microcantilever of the chemical sensor. The calixarene has the formula:

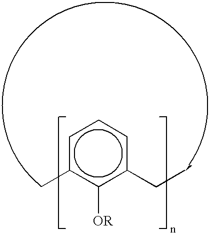

and includes conformational isomers thereof, wherein n is 4 to 12, and wherein R is any atom or group of atoms provided that at least one R moiety is (mercaptoalkyl)-substituted alkyl. Two of the R moieties together may comprise a crown ether as shown in the molecule bonded to the gold layer in FIG. 6. Non-limiting examples include 25,27-bis[11 (mercaptodecyl)undecyloxy]calix[4]arene-crown-5 and 25,27-bis[11 (mercaptodecyl)undecyloxy]calix[4]arene-crown-6. Alternatively, one or more R moieties may include a urea group as shown in the last structure in FIG. 8 which has two urea groups. Preferably, in either form, at least one R moiety is (mercapto-C₅-C₂₅ alkyl)-substituted C₅-C₂₅ alkyl. For example, two (mercapto-decyl)-undecyl groups as shown in the molecule bonded to the gold layer in FIG. 6.

Another method of the invention uses an alternative calixarene that may be bonded to the silicon surface on the microcantilever of the chemical sensor. The calixarene has the formula:

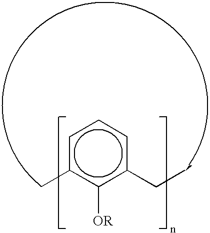

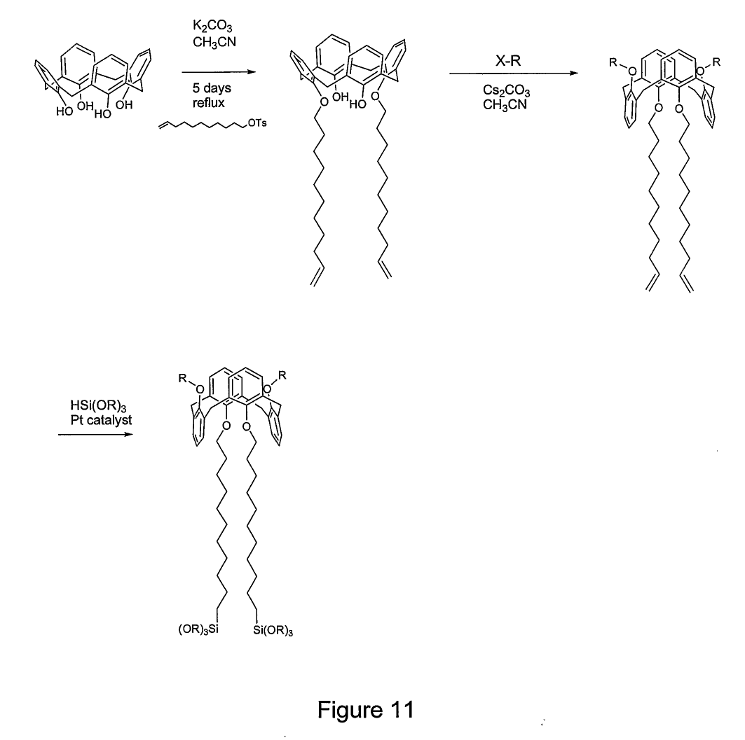

and includes conformational isomers thereof, wherein n is 4 to 12, and wherein R is any atom or group of atoms provided that at least one R moiety is R₁—Si(OR₂)₃ wherein R₁ is alkylene and R₂ is any atom or group of atoms. Preferably, R₁ is C₅-C₂₅ alkylene, and R₂ is hydrogen or alkyl. One example of this calixarene is shown in the last structure in FIG. 11.

In a method for forming a chemical sensor according to the invention, an attachment group is chemically bonded to a chemical recognition agent. The attachment group includes a first spacer group chemically bonded to a bridging atom and a second spacer group chemically bonded to the bridging atom. The first spacer group is chemically bonded to the chemical recognition agent. The bridging atom is then chemically bonded to a metallic coating disposed on a side of a microcantilever. In one version of the method, an alkenyl group is chemically bonded to the chemical recognition agent, and a mercaptoalkyl group is chemically bonded to the double bond of the alkenyl group such that the bridging atom is sulfur, the first spacer group is an alkylene group, and the second spacer group is an alkyl group. The chemical recognition agent may be a calixarene bonded to a group selected from crown ethers, ureas, thioureas, and cationic ion exchangers. Preferably, the alkenyl group is a C₅-C₂₅ alkenyl group, and the mercaptoalkyl group is a (mercapto-C₅-C₂₅ alkyl) group. For example, the mercaptoalkyl group is mercapto-decyl, and the alkenyl group is undecylene.

In an example of this method of the invention, a terminally substituted alkene is reacted with the chemical recognition agent, and an alkanethiol is reacted with the double bond of the alkene such that the bridging atom is sulfur, the first spacer group is an alkylene group, and the second spacer group is an alkyl group. Preferably, the substituted alkene is a substituted C₅-C₂₅ alkene (e.g., 10-undecen-1-tosylate), and the alkanethiol is a C₅-C₂₅ alkanethiol (e.g., decanethiol).

In another method for forming a chemical sensor according to the invention, the silicon surface of a microcantilever is processed to provide terminal hydrogen groups, and photochemical hydrosilylation is used to carbon link a chemical recognition agent to the silicon surface. Preferably, the chemical recognition agent comprises a calixarene. In one version, the calixarene has an alkenyl group, and the alkenyl group is carbon linked to the silicon surface. Preferably, the alkenyl group is a C₅-C₂₅ alkenyl group. The chemical recognition agent may be a calixarene bonded to a group selected from crown ethers, ureas, thioureas, and cationic ion exchangers.

In yet another method for forming a chemical sensor according to the invention, a chemical recognition agent is bonded to the silicon surface of a microcantilever having an oxidized, hydrated silicon surface. The chemical recognition agent includes at least one terminal R group wherein R is R₁—Si(OR₂)₃ and wherein R₁ is alkylene and R₂ is any atom or group of atoms. The chemical recognition agent may comprise a calixarene having the formula:

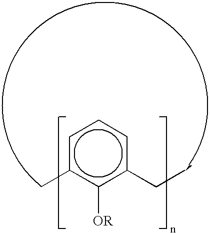

and conformational isomers thereof,
wherein n is 4 to 12, and R is any atom or group of atoms provided that at least one R moiety is R₁—Si(OR₂)₃ wherein R₁ is alkylene and R₂ is any atom or group of atoms. Preferably, R₁ is C₅-C₂₅ alkylene and R₂ is hydrogen or alkyl.

### EXAMPLES

The following Examples have been presented in order to further illustrate the invention and are not intended to limit the invention in any way. Below are illustrated the preparation of a Cs⁺ selective reagent, and a reagent selective for the sorption of nitro containing explosives.

### Example 1

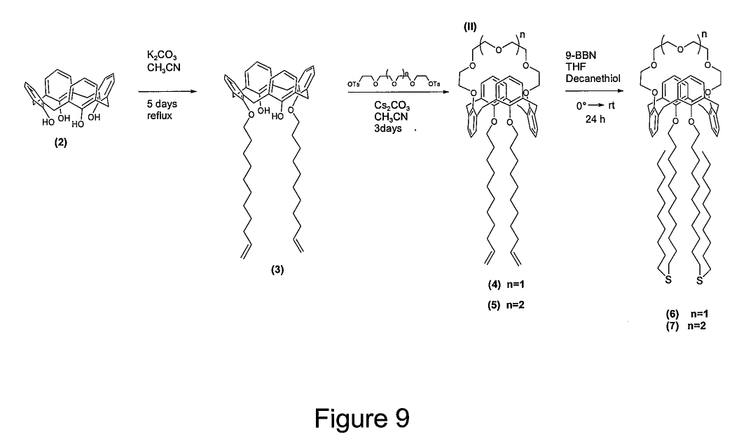

A synthetic strategy is shown in FIG. 9 and starts with the selective alkylation of calix[4]arene (2) in the 24,26-position with 10-undecen-1-tosylate. It was found that using 11-bromo-1-undecen as alkylating agent yielded a lot of side products and a hard to separate mixture of di- and tetra substituted calixarenes. The crowning was carried out using a procedure of Casnati et al., J. Am. Chem. Soc. 1995, 117, 2767, using tetraethyleneglycolditosylate and pentaethyleneglycolditosylate, respectively, to obtain 1,3-Bis-11-undecyloxy-calix[4]arene-crown-5 (4) and 1,3-bis-11-undecyloxy-calix[4]arene-crown-6 (5). The ¹H-NMR measurements of the ArCH₂Ar protons confirmed that the calixarenes (4-7) have a rigid 1,3 alternate conformation (see, Jaime et al., J. Org. Chem. 1991, 56, 3372). Subsequently, (4, 5) were converted to the dialkylsulfide adsorbates (6, 7) by selective addition of decanethiol to the double bond using 9-borabicyclo[3.3.1]nonane (9-BBN) (see, Masuda et al., Chem. Comm. 1991, 1444). The ¹H-NMR spectra of (6) and (7) showed the complete conversion of the double bond.

### Example 2

### 25,27-bis(11-undecenyloxy)-26,28-dihydroxycalix[4]arene (3)

A suspension of calix[4]arene (2.12 g, 5 mmol), 10-undecen-1-tosylate (3.4 g, 10.5 mmol), and potassium carbonate (1.4 g, 10.1 mmol) in 50 ml acetonitrile was heated with stirring under argon for 5 days. The solvent was removed under reduced pressure, and the residue was partitioned between chloroform and 1 N HCl. The organic layer was washed with 1 N HCl and brine, dried over NaSO₄ and evaporated to give a tan oil. The oil was extracted several times with boiling methanol. The solution was allowed to cool down to 0° C., and a white solid precipitated. The results were:

1.64 g(45.3%)

C₅₀H₆₄O₄ (729.04)

¹H-NMR (CDCl₃): δ 8.23 (s, 2H, OH); 7.02 (d, 4H, m-ArH); 6.90 (d, 2H, o-ArH); 6.73 (t, 2H, o-ArH); 6,63 (t, 2H, o-ArH); 5.79 (m, 2H, CH═); 4.95 (m, 4H, CH₂═); 4.30 (d, 4H, ArCH₂Ar); 3.98 (t, 4H, OCH₂); 3.35 (d, 4H, ArCH₂Ar); 3.35 (m, 8H, OCH₂CH₂— and CH₂═CHCH₂); 2.15-1.54 (m, 12H, chain)

¹³C-NMR (CDCl₃): δ 153.32 (ArC—OH); 151.97 (ArC-OR); 139.20 (CH═); 133.45 (oArC); 128.85 (o-ArC); 128.36 (m-ArC); 128.15 (m-ArC); 125.22 (p-ArC); 118.90 (P—ArC); 114.1 (CH₂═); 76.68 (OCH₂); 33.81 (CH₂═CHCH₂—); 31.41 (ArCH₂Ar); 29.99 (CH₂═CHCH₂CH₂—); 29.61 (CH₂═CHCH₂CH₂CH₂—); 29.56 (OCH₂CH₂); 29.50 (OCH₂ CH₂CH₂); 29.17 ((CH₂═CHCH₂ CH₂CH₂CH₂—); 28.96 (OCH₂ CH₂ CH₂CH₂); 25.95 (OCH₂CH₂CH₂CH₂CH₂).

### Example 3

### General Procedure for Synthesis of 1,3-alt-bis-(alkoxy)-calix[4]arene-crown-n (n=5.6)

A solution of 1,3-bis-(alkoxy)calix[4]arene (1.1 g, 1.58 mmol), Cs₂CO₃ (2.93 g, 9.3 mmol), and tetra- or pentaethyleneglycol di-p-toluenesulfonate (828 mg, 1.65 mmol) in dry MeCN (200 ml) was refluxed under slightly pressure for 5 days. Subsequently, the solvent was removed under reduced pressure. The residue was dissolved in CH₂Cl₂ and washed with 1 N HCl, water and brine. The organic phase was separated, dried over NaSO₄ and evaporated to afford a yellow oil, which was purified by column chromatography (EtOAc/hexanes 1:1 to EtOAc 100%-gradient). The results were:

### A. 1,3-Alternate 25,27-bis(10-undecenyloxy)calix[4]arene-crown-5 (4) C₆₀H₈₂O₈ (931.29)

¹H-NMR (CDCl₃): δ 7.07 (d, 4H, m-ArH); 7.00 (d, 4H, m-ArH); 6.81 (d, 2H, o-ArH); 6.73 (t, 2H, o-ArH); 5.78 (m, 2H, CH═); 4.96 (m, 4H, CH₂═); 3.84 (s, 8H, ArCH₂Ar); 3.57 (m, 8H, ArO(CH₂CH₂O)₂CH₂, ArOCH₂(CH₂)₈CH═CH₂); 3.36 (m, 8H, ArOCH₂CH₂OCH₂—); 3.13 (t, 4H, ArOCH₂CH₂OCH₂CH₂—); 2.05 (q, 4H, OCH₂ (CH₂)₇ CH₂CH═CH₂); 1.38-1.15 (m, 14H, chain)

¹³C-NMR (CDCl₃): δ 156.91 (ArC-OH); 156.47 (ArC-OR); 139.19 (CH═); 134.05, 133.71 (o-ArC); 129.76, 129.60 (m-ArC); 122.09 (p-ArC); 114.14 (CH₂=); 71.18, 71.06, 70.98, 70.59, 69.87 (OCH₂); 37.89 (ArCH₂Ar), 33.81 (CH₂═CHCH₂); 29.71 (CH₂═CHCH₂CH₂—); 29.69 (CH₂═CHCH₂CH₂CH₂—); 29.55 (OCH₂CH₂); 29.28 (OCH₂CH₂CH₂); 29.22 (CH₂═CHCH₂CH₂CH₂CH₂—); 28.98 (OCH₂CH₂CH₂CH₂); 25.79 (OCH₂CH₂CH₂CH₂CH₂).

### B. 1,3-Alternate 25,27-bis(10-undecenyloxy)calix[4]arene-crown-6 (5) C₅₈H₇₈O₇ (887.24)

¹H-NMR (CDCl₃): δ 7.07 (d, 4H, m-ArH); 7.00 (d, 4H, m-ArH); 6.86 (d, 2H, o-ArH); 6.78 (t, 2H, o-ArH); 5.82 (m, 2H, CH═); 4.99 (m, 4H, CH₂═); 3.83 (s, 8H, ArCH₂Ar); 3.56 (m, 8H, ArOCH₂CH₂O—, ArOCH₂(CH₂)₈CH═CH₂); 3.36 (m, 8H, ArOCH₂CH₂OCH₂CH₂O—, ArOCH₂CH₂OCH₂CH₂O—); 3.12 (t, 4H, ArOCH₂CH₂OCH₂CH₂O—); 2.16 (q, 4H, OCH₂(CH₂)₇CH₂CH═CH₂); 1.48-1.15 (m, 14H, chain)

¹³C-NMR (CDCl₃): δ 156.91 (ArC-OH); 156.47 (ArC-OR); 139.19 (CH═); 134.05, 133.71 (o-ArC); 129.76, 129.60 (m-ArC); 122.09 (p-ArC); 114.14 (CH₂=); 76.67, 71.18, 71.06, 70.98, 70.59, 69.87 (OCH₂); 37.89 (ArCH₂Ar), 33.81 (CH₂═CHCH₂); 29.71 (CH₂═CHCH₂CH₂—); 29.69 (CH₂═CHCH₂CH₂CH₂—); 29.55 (OCH₂CH₂); 29.28 (OCH₂CH₂CH₂); 29.22 (CH₂═CHCH₂CH₂CH₂CH₂—); 28.98 (OCH₂CH₂CH₂CH₂); 25.79 (OCH₂CH₂CH₂CH₂CH₂).

### Example 4

### General Procedure for Addition of Thiols to Double Bonds

The alkene compound (0.01 mol) and 0.015 mol decanethiol were dissolved in 15 ml dry THF and cooled to 0° C. After addition of 0.5 ml 9-BBN solution (0.1 m in THF) the reaction mixture was stirred 1 hour at 0° C. and at room temperature overnight. After the addition of 3 ml H₂O to destroy the excess of boron, the solvent was removed under reduced pressure. The crude product was dissolved in methylenechloride and washed with water. After drying over NaSO₄, filtration, and evaporation, the residue was washed 2 times with ether (to remove excess of decanethiol) to give white, waxy highly hygroscopic solids. The compounds were dried in high vacuum and stored over CaCl₂. The results were:

### A. 25,27-bis[11 (mercaptodecyl)undecyloxy]calix[4]arene-crown-5 (6) C₇₈H₁₂₂O₇S₂ (1235.93)

¹H-NMR (CDCl₃): δ 7.07 (d, 4H, m-ArH); 7.00 (d, 4H, m-ArH); 6.86 (d, 2H, o-ArH); 6.78 (t, 2H, o-ArH); 3.82 (s, 8H, ArCH₂Ar); 3.55 (m, 8H, ArOCH₂CH₂O—, ArOCH₂(CH₂)₈CH═CH₂); 3.36 (m, 8H, ArOCH₂CH₂OCH₂CH₂O—, ArOCH₂CH₂OCH₂CH₂O—); 3.12 (t, 4H, ArOCH₂CH₂OCH₂CH₂O—); 2.16 (q, 4H, OCH₂(CH₂)₇CH₂CH═CH₂); 1.48-1.15 (m, 14H, chain) 3.36 (m, 8H, ArOCH₂CH₂OCH₂—); 3.13 (t, 4H, ArOCH₂CH₂OCH₂CH₂—); 2.05 (q, 4H, OCH₂ (CH₂)₇ CH₂CH═CH₂); 1.38-1.15 (m, 14H, chain)

¹³C-NMR (CDCl₃): δ 156.96, 156.09 (ArC-O); 134.20, 134.03 (o-ArC); 129.68, 129.31 (m-ArC); 129.18, 122.32 (p-ArC); 72.65, 70.57, 70.29, 69.94, 68.32 (OCH₂); 38.17 (ArCH₂Ar), 32.21, 31.87, 30.33; 30.09; 29.70, 29.53, 29.29, 29.21, 29.1, 28.96, 28.51, 25.76; 22.65; 14.10 (CH₃)

### B. 25,27-bis[11(mercaptodecyl)undecyloxy]calix[4]arene-crown-6 (7) C₈₀H₁₂₆O₈S₂ (1279.98)

¹H-NMR (CDCl₃): δ 7.07 (d, 4H, m-ArH); 7.00 (d, 4H, m-ArH); 6.86 (d, 2H, o-ArH); 6.78 (t, 2H, o-ArH); 5.82 (m, 2H, CH═); 4.99 (m, 4H, CH₂═); 3.83 (s, 8H, ArCH₂Ar); 3.56 (m, 8H, ArOCH₂CH₂O—, ArOCH₂(CH₂)₈CH═CH₂); 3.36 (m, 8H, ArOCH₂CH₂OCH₂CH₂O—, ArOCH₂CH₂OCH₂CH₂O—); 3.12 (t, 4H, ArOCH₂CH₂OCH₂CH₂O—); 2.16 (q, 4H, OCH₂(CH₂)₇CH₂CH═CH₂); 1.48-1.15 (m, 14H, chain)

¹³C-NMR (CDCl₃): δ 156.91, 156.47 (ArC-O); 134.04, 133.71 (o-ArC); 129.76, 129.61 (m-ArC); 122.08 (p-ArC); 71.19, 71.06, 70.99, 70.61, 69.88 (OCH₂); 37.88 (ArCH₂Ar), 32.19, 31.87, 29.75, 29.72, 29.65, 29.54, 29.36, 29.30, 29.02, 28.96, 25.81, 22.66 (SCH₂); 14.10 (CH₃).

### Example 5

Photochemical hydrosilylation can be used for the direct covalent attachment of the 1,3-Alternate 25,27-bis(10-undecenyloxy)calix[4]arene-crown-5 (4) and/or 1,3-Alternate 25,27-bis(10-undecenyloxy)calix[4]arene-crown-6 (5) of Example 3 and FIG. 9 via robust Si—C bonds to the silicon surface of a microcantilever. This can be achieved in a two-step process involving hydrogen termination of the silicon cantilever surface and subsequent photochemical (UV) hydrosilylation with the terminal double bonds of the 1,3-Alternate 25,27-bis(10-undecenyloxy)calix[4]arene-crown-5 (4) and/or 1,3-Alternate 25,27-bis(10-undecenyloxy)calix[4]arene-crown-6 (5), which results in stable Si—C surface linkages (see a general reaction scheme in FIG. 10).

Hydrogen termination of the silicon surface can be achieved by immersing each cantilever (˜6 min) in 40% aqueous NH₄F solution, which is purged with argon for at least 30 minutes to remove dissolved oxygen. The resulting surface (Si—H) can be dried in an argon flow and evacuated to remove any residual NH₄F. Each hydrogen-terminated silicon microcantilever can be placed in a quartz tube (2 mm. i.d.) and transferred under argon backflow into the second compartment of the quartz cell. Then all cantilevers can be evacuated together with the 1,3-Alternate 25,27-bis(10-undecenyloxy)calix[4]arene-crown-5 (4) and/or 1,3-Alternate 25,27-bis(10-undecenyloxy)calix[4]arene-crown-6 (5). The silicon surface can be irradiated using frequencies emitted by a mercury lamp (100 W, ˜25 cm distance from the surface) for 7-10 days to ensure sufficient time for dense packing of the hydrocarbon chains.

### Example 6

The 25,27-bis[11 (mercaptodecyl)undecyloxy]calix[4]arene-crown-5 (6) and 25,27-bis[11 (mercaptodecyl)undecyloxy]calix[4]arene-crown-6 (7) of Example 4 can be anchored to the gold surface of a microcantilever using the technique of Bain et al., J. Am. Chem. Soc., 1989, 111, 321, to form the microcantilever of FIG. 6.

### Example 7

### Functionalization of calix[4]arene with 3-bromopropyl-phthalimide

Nitro-groups are weak hydrogen-bond acceptors, which interact only weakly with each other (see, Wozniak et al., J. Phys. Chem., 1994, 98, 13755). However, combined with a strong hydrogen-bond donor, they are able to show significant interactions (see, Graham et al., New. J. Chem. 2004, 28, 161). NH groups of ureas show strong hydrogen acidity and are known to build planar networks based on H-bonding between the carbonyl oxygen and the amino-hydrogen. We functionalized the recognition site of the calix[4]arene with 3-bromopropyl-phthalimide, which after hyrazinolyzation yields the bis-amino compound, which subsequently is transformed to the bis-urea calixarene using 4-t-butylbenzene isocyanate. See FIG. 8. We expect the distance of the two ureas on the calixarene to be large enough to prevent to strong interactions, and allow the nitrobenzene derivatives to interact sufficiently with the monolayer. Without intending to be bound by theory, it is conceivable that geometrical reasons assist the interaction between the planar aromatic ring and the preferred planar orientation of H-bonding urea units and disturb intramolecular interactions.

### Example 8

The chemistry of attachment can be modified so that a siloxane reagent can be added to the olefin on the calixarene. The olefin can be modified with a hydrosilane reagent so that Si(OR)₃ is added to the calixarene. Preferably, R is hydrogen or alkyl. The chemistry is shown in FIG. 11. The Si(OR)₃ terminal group of the calixarene can be hydrolyzed and covalently bonded to an oxidized, hydrated silicon surface to form a siloxane bridging group between the silicon surface and the alkyl spacer group shown in FIG. 11.

Thus, the invention provides methods for the preparation of stable, self-assembled monolayers on the silicon surface or the gold surface of gold coated microcantilevers so that the microcantilevers can be used in chemical sensing applications. Although the single cantilever approach works well in laboratory applications, it is less useful in real environment applications where many other parameters can produce signal interference. To avoid this potential problem, it is best to look at the differential response of an array of cantilevers. For example, variations in physical parameters such as temperature, acceleration, and mechanical noises can contribute to cantilever bending. Differential signals obtained by common mode rejection can provide highly sensitive data.

Chemical selectivity can be achieved by arrays consisting of several microcantilevers, each coated with different selective or partially selective coatings. The response of a given modified microcantilever will depend on the concentration of the analyte and the strength of the coating-analyte interactions (e.g. hydrogen bonding, dispersion, and dipole-dipole interactions). A unique response pattern characteristic to a particular analyte can be obtained from an array where each microcantilever is modified with a different coating. The higher the number of modified cantilevers, the greater the uniqueness of the response pattern. Since the microcantilever response to a given analyte depends on the functional end-groups of modifying agents, judicious selection of coatings can lead to significant differences in the response patterns for different analytes. Using an array consisting of a large number of microcantilevers, unique response patterns can be attained for individual analytes, class of analytes, or analytes in complex mixtures. The results of testing with a large number of analyte and mixtures are recorded in a look-up table and referenced routinely when an array is in service.

Although the present invention has been described in considerable detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

### INDUSTRIAL APPLICABILITY

The invention relates to methods for the preparation of stable, self-assembled monolayers on the silicon surface or gold surface of gold coated microcantilevers that can be used in chemical sensing applications.

What is claimed is:

1. A chemical sensor comprising:
a microcantilever;
a metallic coating disposed on a side of the microcantilever;
a bridging atom bonded to the metallic coating;
a first spacer group bonded to the bridging atom;
a second spacer group bonded to the bridging atom; and
a chemical recognition agent for detecting an atom, a molecule or an ion, the chemical recognition agent being bonded to the first spacer group.

2. The chemical sensor of claim 1 wherein:
the metallic coating comprises a metal selected from the group consisting of gold, platinum, copper, palladium, aluminum and titanium.

3. The chemical sensor of claim 1 wherein:
the metallic coating comprises gold, and
the bridging atom is sulfur.

4. The chemical sensor of claim 1 wherein:
the first spacer group is selected from the group consisting of unsubstituted or substituted alkylene groups, unsubstituted or substituted alkenylene groups, or unsubstituted or substituted alkynylene groups.

5. The chemical sensor of claim 1 wherein:
the first spacer group is selected from C₅-C₂₅ alkylene groups.

6. The chemical sensor of claim 1 wherein:
the second spacer group is selected from the group consisting of unsubstituted or substituted alkyl groups, unsubstituted or substituted alkenyl groups, or unsubstituted or substituted alkynyl groups.

7. The chemical sensor of claim 1 wherein:
the second spacer group is selected from C₅-C₂₅ alkyl groups.

8. The chemical sensor of claim 1 wherein:
the chemical recognition agent comprises a chemical recognition agent selected from quaternary ammonias, pyiridines, crown ethers, azacrown compounds, borate esters, ureas, thioureas, antibody-antigens, organic acids, organic esters, organic amides, organic amines, organic aldehydes, phosphonic acids, phosphonic esters, buckyballs, and hydroxyls.

9. The chemical sensor of claim 1 wherein:
the chemical recognition agent comprises a calixarene bonded to a group selected from crown ethers, ureas, thioureas, and cationic ion exchangers.

10. The chemical sensor of claim 1 wherein:
the metallic coating comprises gold,
the bridging atom is sulfur,
the first spacer group is selected from C₅-C₂₅ alkylene groups,
the second spacer group is selected from C₅-C₂₅ alkyl groups, and
the chemical recognition agent comprises a calixarene bonded to a crown ether.

11. The chemical sensor of claim 1 wherein:
the metallic coating comprises gold,
the bridging atom is sulfur,
the first spacer group is selected from C₅-C₂₅ alkylene groups,
the second spacer group is selected from C₅-C₂₅ alkyl groups, and
the chemical recognition agent comprises a calixarene bonded to a urea.

12. The chemical sensor of claim 1 further comprising:
means for detecting a binding interaction between the chemical recognition agent and the atom, the molecule or the ion.

13. The chemical sensor of claim 12 wherein the means for detecting the binding interaction comprises at least one method selected from the group consisting of optical, piezoresistive, piezoelectric, and capacitive.

14. The chemical sensor of claim 12 wherein the binding interaction is reversible using electrocycling or electrolytecycling.

15. The chemical sensor of claim 12 wherein the binding interaction causes a change in surface stress in the microcantilever.

16. The chemical sensor of claim 1 comprising:
an array of microcantilevers, at least some of the microcantilevers including a metallic coating disposed on a side of the microcantilever, a bridging atom bonded to the metallic coating, a first spacer group bonded to the bridging atom, a second spacer group bonded to the bridging atom, and a chemical recognition agent for detecting an atom, a molecule or an ion, the chemical recognition agent being bonded to the first spacer group.

17. The chemical sensor of claim 16 wherein:
at least two of the microcantilevers have different chemical recognition agents.

18. The chemical sensor of claim 16 further comprising:
at least one reference microcantilever.

19. A chemical sensor comprising:
a microcantilever having a silicon surface and a surface having a metallic coating;
a spacer group bonded to the silicon surface;
a chemical recognition agent for detecting an atom, a molecule or an ion, the chemical recognition agent being bonded to the spacer group,
wherein the chemical recognition agent comprises a calixarene.

20. The chemical sensor of claim 19 wherein:
the metallic coating comprises a metal selected from the group consisting of gold, platinum, copper, palladium, aluminum and titanium.

21. The chemical sensor of claim 19 wherein:
the metallic coating comprises gold.

22. The chemical sensor of claim 19 wherein:
the spacer group is selected from the group consisting of unsubstituted or substituted alkylene groups, unsubstituted or substituted alkenylene groups, or unsubstituted or substituted alkynylene groups.

23. The chemical sensor of claim 19 wherein:
the spacer group is selected from C₅-C₂₅ alkylene groups.

23. The chemical sensor of claim 19 wherein:
the chemical recognition agent comprises a calixarene bonded to a group selected from crown ethers, ureas, thioureas, and cationic ion exchangers.

24. The chemical sensor of claim 19 wherein:
the chemical recognition agent comprises a calixarene bonded to a crown ether.

25. The chemical sensor of claim 19 wherein:
the chemical recognition agent comprises a calixarene bonded to a urea.

26. The chemical sensor of claim 19 further comprising:
means for detecting a binding interaction between the chemical recognition agent and the atom, the molecule or the ion.

27. The chemical sensor of claim 26 wherein the means for detecting the binding interaction comprises at least one method selected from the group consisting of optical, piezoresistive, piezoelectric, and capacitive.

28. The chemical sensor of claim 26 wherein the binding interaction is reversible using electrocycling or electrolytecycling.

29. The chemical sensor of claim 26 wherein the binding interaction causes a change in surface stress in the microcantilever.

30. The chemical sensor of claim 19 comprising:
an array of microcantilevers.

31. The chemical sensor of claim 30 wherein:
at least two of the microcantilevers have different chemical recognition agents.

32. The chemical sensor of claim 30 further comprising:
at least one reference microcantilever.

33. The chemical sensor of claim 19 wherein:
the spacer group includes a siloxane group bonded to the silicon surface.

34. A calixarene having the formula: 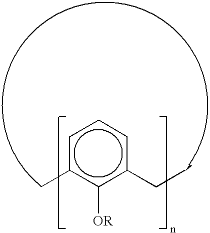
and conformational isomers thereof,
wherein n is 4 to 12, and wherein R is any atom or group of atoms provided that at least one R moiety is (mercaptoalkyl)-substituted alkyl.

35. The calixarene of claim 34 wherein:
two R moieties together comprise a crown ether.

36. The calixarene of claim 34 wherein:
at least one R moiety includes a urea group.

37. The calixarene of claim 34 wherein:
at least one R moiety is (mercapto-C₅-C₂₅ alkyl)-substituted C₅-C₂₅ alkyl.

38. The calixarene of claim 34 wherein the calixarene is
25,27-bis[11 (mercaptodecyl)undecyloxy]calix[4]arene-crown-5.

39. The calixarene of claim 34 wherein the calixarene is
25,27-bis[11 (mercaptodecyl)undecyloxy]calix[4]arene-crown-6.

40. A calixarene having the formula: 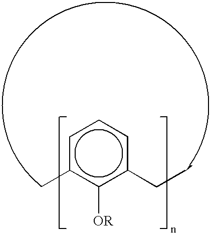
and conformational isomers thereof,
wherein n is 4 to 12, and R is any atom or group of atoms provided that at least one R moiety is R₁—Si(OR₂)₃ wherein R₁ is alkylene and R₂ is any atom or group of atoms.

41. The calixarene of claim 40 wherein R₁ is C₅-C₂₅ alkylene.

42. The calixarene of claim 40 wherein R₂ is hydrogen or alkyl.

43. A method for forming a chemical sensor, the method comprising:
(a) chemically bonding an attachment group to a chemical recognition agent, the attachment group comprising a first spacer group chemically bonded to a bridging atom and a second spacer group chemically bonded to the bridging atom, the first spacer group being chemically bonded to the chemical recognition agent; and
(b) chemically bonding the bridging atom to a metallic coating disposed on a side of a microcantilever.

44. The method of claim 43 wherein step (a) comprises:
chemically bonding an alkenyl group to the chemical recognition agent, and
chemically bonding a mercaptoalkyl group to the double bond of the alkenyl group such that the bridging atom is sulfur, the first spacer group is an alkylene group, and the second spacer group is an alkyl group.

45. The method of claim 44 wherein:
the chemical recognition agent comprises a calixarene bonded to a group selected from crown ethers, ureas, thioureas, and cationic ion exchangers.

46. The method of claim 44 wherein:
the alkenyl group is a C₅-C₂₅ alkenyl group, and
the mercaptoalkyl group is a (mercapto-C₅-C₂₅ alkyl) group.

47. The method of claim 43 wherein step (a) comprises:
reacting an alkene with the chemical recognition agent, and
reacting an alkanethiol with the double bond of the alkene such that the bridging atom is sulfur, the first spacer group is an alkylene group, and the second spacer group is an alkyl group.

48. The method of claim 47 wherein:
the alkene is a C₅-C₂₅ alkene, and
the alkanethiol is a C₅-C₂₅ alkanethiol.

49. The method of claim 47 wherein:
the alkene is a terminally substituted C₅-C₂₅ alkene, and
the alkanethiol is a C₅-C₂₅ alkanethiol.

50. The method of claim 49 wherein:
the double bond of the alkene is in a terminal position.

51. A method for forming a chemical sensor, the method comprising:
hydrogen terminating a silicon surface of a microcantilever; and
carbon linking a chemical recognition agent to the silicon surface using photochemical hydrosilylation,
wherein the chemical recognition agent comprises a calixarene.

52. The method of claim 51 wherein:
the calixarene has an alkenyl group, and
the alkenyl group is carbon linked to the silicon surface.

53. The method of claim 52 wherein:
the alkenyl group is a C₅-C₂₅ alkenyl group

54. The method of claim 51 wherein:
the chemical recognition agent comprises a calixarene bonded to a group selected from crown ethers, ureas, thioureas, and cationic ion exchangers.

55. A method for forming a chemical sensor, the method comprising:
providing a microcantilever having an oxidized, hydrated silicon surface; and
bonding a chemical recognition agent to the silicon surface,
wherein the chemical recognition agent includes a terminal R group
wherein R is R₁—Si(OR₂)₃ wherein R₁ is alkylene and R₂ is any atom or group of atoms.

56. The method of claim 55 wherein:
the chemical recognition agent comprises a calixarene having the formula: 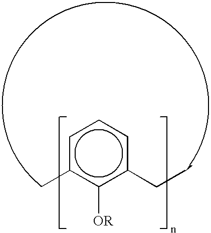
and conformational isomers thereof,
wherein n is 4 to 12, and R is any atom or group of atoms provided that at least one R moiety is R₁—Si(OR₂)₃ wherein R₁ is alkylene and R₂ is any atom or group of atoms.

57. The method of claim 56 wherein R₁ is C₅-C₂₅ alkylene.

58. The method of claim 56 wherein R₂ is hydrogen or alkyl.
